(19) United States
(12) Patent Application Publication
(10) Pub. No.: US 2025/0049236 A1
(43) Pub. Date: Feb. 13, 2025

(54) Bedding Set

(71) Applicant: Shakera Austion, Bronx, NY (US)

(72) Inventor: Shakera Austion, Bronx, NY (US)

(21) Appl. No.: 18/446,992

(22) Filed: Aug. 9, 2023

(51) Int. Cl. A47G 9/02; A45C 13/10; A61L 2/10; A61L 2/22

(52) U.S. Cl. CPC A47G 9/0246; A45C 13/10; A47G 9/0253; A47G 9/0261; A61L 2/10; A61L 2/22; A45C 2013/1015; A47G 2400/022

(57) ABSTRACT

The present invention relates generally to the field of bedding configurations. More specifically, the present invention relates to an improved bedding set that has at least one pillowcase, a duvet, a top sheet, a fitted sheet, a lining, a protective plastic layer, and a transportation bag that contains the ability to treat the bedding set for germs, viruses and bugs. At least two components of the bedding set may have a fastener assembly that allows each component to remain attached to one another such that the set needs not be re-made after a night of sleep. Further, the disinfection system within the transport bag contains a UV disinfection light and sprayer that receives a cartridge that receives a solution for treating the bedding.

### FIELD OF THE INVENTION

The present invention relates generally to the field of bedding linens and other coverings. More specifically, the present invention relates to an improved bedding set that is comprised of at least one pillowcase, a duvet, a top sheet, a fitted sheet, a lining, a protective plastic layer and a transportation bag that contains a disinfectant material. The bottom and/or top surface of each component may further include a snap button or other fastener assembly or the like that allows each component to remain attached to one another, such that the set needs not be re-made after a night of sleep. Further, the disinfectant material within the transport bag includes a UV disinfection light and sprayer that receives a cartridge that contains bed bug killing or repelling chemicals and other antibacterial or antiviral agents. As a result, the UV light and sprayer kills bed bugs, pathogens, bacteria, and virus that may be on the bedding set after it is used. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

Many individuals travel and stay in locations such as hotels, motels, Airbnbs, hostels, etc. while traveling. The locations normally provide a bed that is complete with bedding materials. However, often times the provided bedding materials are unclean or the individual may not want to use sheets and other bedding materials that have been previously slept in by other travelers. Further, the bedding materials may contain bed bugs, or other pests which can then transfer to an individual's clothes or belongings after sleeping in the bedding, and be brought back to the individual's home. In such situations, a bed bug infestation could occur in the individual's home which costs upwards of thousands of dollars to exterminate, as well as the inconvenience of having to remove the vermin. In addition, individuals may simply be more comfortable sleeping with their own bedding while traveling, as the provided bedding may be low quality and uncomfortable. However, bedding sets are typically extremely difficult to carry and transport due to their numerous parts, bulkiness and weight. In addition, individuals may wish to bring their bedding to multiple destinations, but do not have the opportunity to clean the bedding after arriving to each different location. Further, bedding may often dislodge in the night during sleep such that it must be remade every morning, which is undesirable for some individuals. In addition, if the bedding moves or is displaced from around the mattress, the individual is exposed to the mattress and any other pre-existing sheets.

Therefore, there exists a long-felt need in the art for an improved bedding set. There also exists a long-felt need in the art for an improved bedding set that is comfortable and ensures the bedding remains sanitary. In addition, there is a long-felt need in the art for an improved bedding set that can be easily transported to and from various locations. Furthermore, there is a long-felt need in the art for a bedding set that does not have to be re-made or otherwise readjusted after a night of sleep.

The subject matter disclosed and claimed herein, in one embodiment thereof, comprises an improved bedding set. The set has a plurality of components including: at least one pillowcase, a duvet, a top sheet, a fitted sheet, a lining, a protective plastic layer and a transportation bag that provides the ability to disinfect the bedding. The bottom and/or top surface of each component may further include a snap button or other fastener assembly such as hook and loop systems, clips or various combinations thereof that allows each component to remain attached to the bedding set.

In this manner, the improved bedding set of the present invention accomplishes all of the forgoing objectives and provides a sanitary and comfortable bedding set. Further, the set can be easily transported and disinfected via the transportation bag. In addition, the snap button fasteners ensure no component can shift out of position during sleep, which then eliminates the need for the bedding to be remade each morning.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The subject matter disclosed and claimed herein, in one embodiment thereof, comprises an improved bedding set that is easily transportable. The set preferably includes a plurality of components such as at least one pillowcase, a duvet, a top sheet, a fitted sheet, a comfortable lining and a protective plastic layer. Each component of the set may be constructed from any suitable fabric or material, whether synthetic, natural or some blend of synthetic and natural fibers. Each component may also have anti-bacterial, anti-viral and anti-microbial properties. In addition, the components may contain insecticides or other chemicals, including natural substances used for killing or repelling bed bugs. Further, the bottom surface of the lining may have a female snap button, and the top surface of the plastic layer is provided with a male snap button that engage one another. In this manner, both components are secured to one another and cannot shift during sleep. It is also contemplated that the bottom surface of the duvet, and the top and bottom surfaces of the top sheet, the fitted sheet, the lining and the plastic layer may also have snap buttons, or any other fastener known or combinations thereof that allow the components to be secured to one another. In this manner, the entire bedding set does not shift while an individual sleeps on the set, and therefore the set does not need to be remade in the morning.

The bedding set may also include a transport bag that stores the set. The bag may be any shape known in the art and may be flexible, semi-rigid or rigid and may be made of a natural, synthetic or combinations of material. The body of the bag may also have a plurality of designs, logos, patterns, etc. and may have a plurality of carrying handles. A UV disinfection light is located in the interior of the bag, and the light is powered by a disposable or rechargeable battery and can be used when the set is inside the bag to kill pathogens, viruses, bacteria, germs and the like. Further, the interior may contain a sprayer that has a cartridge which holds and dispenses various insecticides and/or cleaning products onto the set. The cartridge may also be used to dispense essential oils in order to create a suitable relaxing atmosphere for the individual.

Accordingly, the improved bedding of the present invention is particularly advantageous as it provides a high-quality bedding set that is easily transportable. Further, the set allows a user to easily disinfect or otherwise treat the set as the individual moves from various locations. In addition, the set does shift during sleep, and does not require the user to re-make the bedding after a night of sleep or nap.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective exploded view of one potential embodiment of an improved bedding set of the present invention in accordance with the disclosed specification;

FIG. 2 shows a front exploded view of one potential embodiment of an improved bedding set of the present invention in accordance with the disclosed structure; and

FIG. 3 provides a perspective view of one potential embodiment a transport bag of an improved bedding set of the present invention in accordance with the disclosed description.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved bedding set. There also exists a long-felt need in the art for an improved bedding set that is comfortable and ensures the bedding remains sanitary, even after repeated uses when laundering is not possible, such as during a long trip. In addition, there is a long-felt need in the art for an improved bedding set that can be easily transported to and from various locations. Further, there exists a long-felt need in the art for a bedding set that does not have to be re-made after a night of sleep.

The present invention, in one exemplary embodiment, is comprised of an improved bedding set that has a plurality of components including at least one pillowcase, a duvet, a top sheet, a fitted sheet, a comfortable lining and a protective plastic layer. Each component of the set may be made from suitable materials, whether natural, synthetic or combinations thereof, and may also have anti-bacterial, anti-microbial and anti-viral properties and insecticides or other solutions used for killing or repelling bed bugs. Further, the bottom surface of the lining may have a female snap button, and the top surface of the plastic layer may have a male snap button that engage one another such that both components are secured to one another to reduce any shifting during sleep. In further differing embodiments, the bottom surface of the duvet, and the top and bottom surfaces of the top sheet, the fitted sheet, the lining and the plastic layer may also have snap buttons or any other fastener known or various combinations of fasteners that allows the bedding components to be secured to one another. In this manner, the entire bedding set does not shift while a user sleeps on the set, and therefore the set need not be remade in the morning. The bedding set may also contain a strap that is insertable beneath the mattress, such that the bedding set can be secured so that the bedding set does not fall off the mattress.

The bedding set may also include a transport bag that stores the set. The bag may be any suitable shape and may be flexible, semi-rigid or rigid and may be made of a natural, synthetic or combinations of material. The body of the bag may also have a plurality of designs, logos, patterns, etc. and may have a plurality of carrying handles. The bag may also have a UV disinfection light located in the interior of the bag that is powered by a disposable or rechargeable battery and can be used when the set is inside the bag to kill pathogens, viruses, bacteria, germs and other contaminants. Further, the interior may contain a sprayer that has a cartridge which holds and dispenses various insecticides, solutions and/or cleaning products onto the set while the set is inside the bag. The bag may also utilize a negative pressure such as a vacuum to help draw the solutions through the bedding in order to effectively treat the bedding materials.

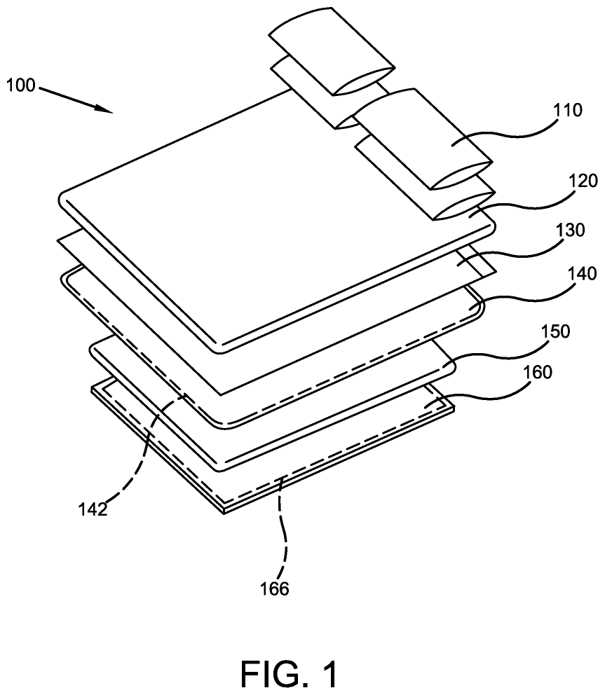

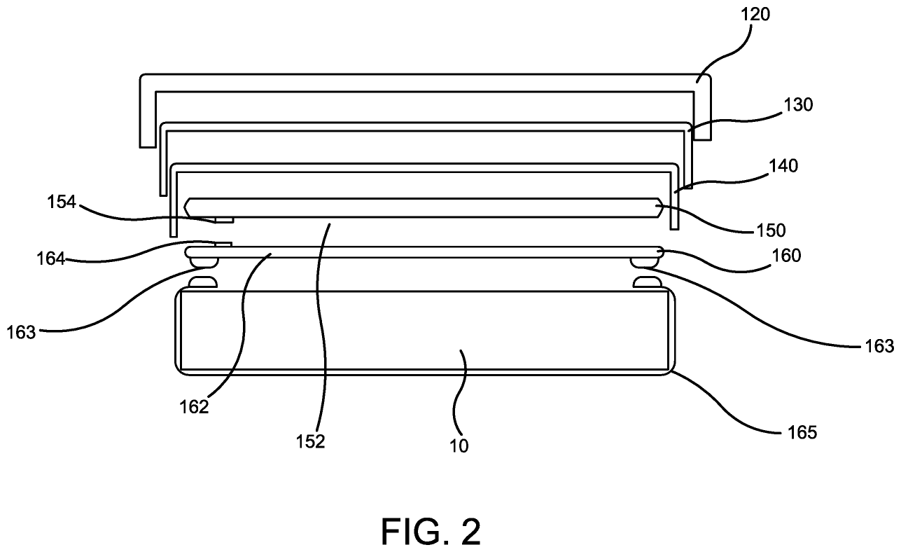

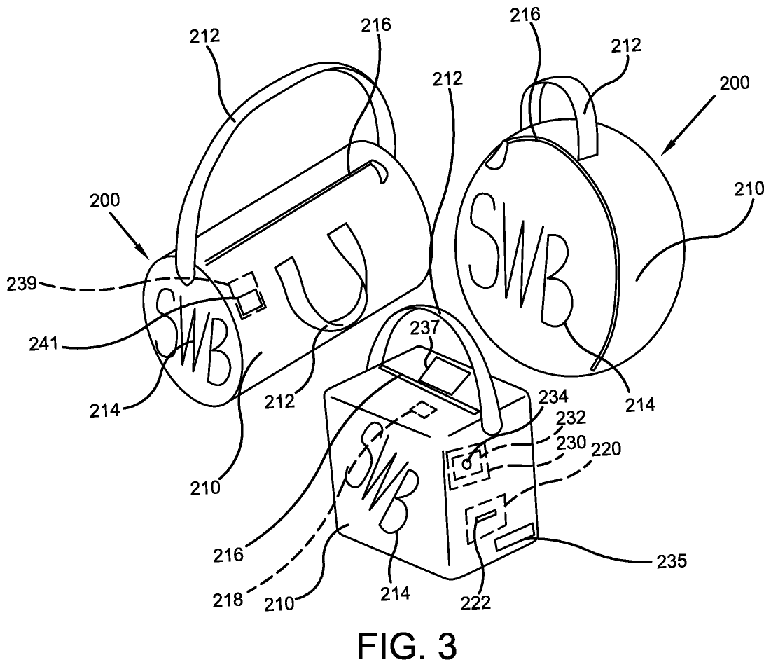

Referring initially to the drawings, FIG. 1 illustrates a perspective exploded view and FIG. 2 illustrates a front exploded view of one potential embodiment of an improved bedding set and carrier of the present invention. The bedding set 100 has a number of components that include but are not limited to: at least one pillowcase 110, a duvet 120, a top sheet 130, a fitted sheet 140, a lining 150, a protective plastic layer 160 and a transport bag 200. The at least one pillowcase 110 may be made of any suitable natural, synthetic or combinations of other materials or fabrics, including but not limited to: cotton, nylon, linen, satin, silk, polyester, etc. or any combination or blends thereof. The fabric of the pillowcase 110 as well as all the linens in the bedding set may also be anti-bacterial, anti-microbial and contain insecticides or other chemicals that kill or repel bed bugs such as a pyrethrin, pyrethroid, desiccant, cold-pressed neem oil, pyrroles, neonicotinoids, essential oils, diatomaceous earth, soapy solutions, vinegar based solutions, alcohol, witch hazel, bay leaves, cedar, citrus oil, chili powder, peppermint oil, spearmint oil, tea tree oil, lemongrass oil, an eucalyptus oil and combinations thereof. A sachet 239 (see FIG. 3) may also be provided that includes one or more of the foregoing 241 to aid in the treating of the bedding set. The duvet 120 may also be made of any natural or synthetic fabric or blends or combinations thereof, including but not limited to: cotton, nylon, linen, satin, silk, polyester, etc. or any combination blend thereof. In addition, the duvet 120 may be filled with down, feathers, silk, wool, microfiber, hollowfiber, other synthetic or natural fillings or combinations thereof.

The top sheet 130 and fitted sheet 140 may be made of any natural, synthetic or blends of fibers, including but not limited to: cotton, nylon, linen, satin, silk, polyester or any combination blend thereof. The fabric may also be anti-bacterial, anti-microbial and contain insecticides that kill or repel bed bugs such as pyrethrins, pyrethroids, desiccants, cold-pressed neem oil, pyrroles, neonicotinoids, etc. In addition, the fitted sheet 140 may contain an internal elastic band 142 that helps retain the fitted sheet 140 around the corners or edges of the bed 10.

The set 100 also contains a comfortable lining 150 in-between the fitted sheet 140 and a protective plastic layer. The lining 150 is preferably made of a plush, comfortable material that may be memory foam or any other type of cushioned natural, synthetic or blends of materials or fabrics. A protective plastic layer 160 sits below the lining 150 and may also contain an internal elastic band 166 to retain the layer 160 around a bed 10. The bottom surface 152 of the lining 150 has at least one female snap button 154 that engages at least one male snap button 164 on the top surface 162 of the plastic layer 160. In this manner, the lining 150 is secured to the plastic layer 160 such that they cannot separate or move. In differing embodiments of the device, the bottom surface of the duvet 120 and the top and bottom surfaces of the top sheet 130, fitted sheet 140, lining 150 and plastic layer 160 may also have at least one set of snap buttons in order to allow each component 120, 130, 140, 150, 160 to remain fastened to one another. In this manner, the components 120, 130, 140, 150, 160 cannot shift during sleep, and as a result the set 100 does not need to be re-made after a night of sleep. In lieu of snap buttons, the top/bottom surface of any component 120, 130, 140, 150, 160 may have at least one of any fastener including but not limited to: hook and loop, adhesive/adhesive strips, magnets integrated within the material/lining of each component 120, 130, 140, 150, 160, a plurality of strings that can be tied to one another, a plurality of zipper assemblies, or various combinations of fasteners. For example, a hook and loop fabric-type fastener assembly may be placed near the top of the bedding where an individual's head may be and hard fasteners such as snaps may be at the other end of the bedding, such as near the individual's feet. FIG. 2 also shows a strap 165 that may be positioned under the mattress 10 and the ends of the straps 165 may have fasteners 163 to allow the strap to be connected to a portion of the bedding to further help in holding the bedding on the mattress.

FIG. 3 illustrates a perspective view of one potential embodiment a transport bag 200 of an improved bedding set 100 of the present invention. The set 100 can be transported using the transport bag 200. In differing embodiments, the body 210 of the bag may be any size and shape known such as but not limited to: spherical, rectangular, square, etc. and may be constructed from any flexible, semi-rigid, or rigid natural or synthetic material or combinations thereof. The surface of the body 210 may also contain any number of indicia 214 that includes but is not limited to: logos, patterns, designs, colors, text, advertisements, initials, monograms, contact information, etc. For case of carrying, the body 210 may also have a plurality of straps 212 in the form of hand-straps and shoulder straps that may be removable or fixedly-attached to the body 210. Further, the body 210 has a zipper assembly 216 that is used to close the body 210 once the bedding set 100 has been placed inside the body 210. However, in differing embodiments the zipper assembly 216 may be replaced in lieu of any other fastener known in the art such as but not limited to: snap button, hook and loop, magnets, strings, or combinations thereof.

The interior area of the body 210 may further include at least one UV disinfection light 218 that kills bacteria, pathogens, viruses, germs, other vermin and the like. The light 218 receives power from a rechargeable battery 220 that is in electrical communication with the light 218. In differing embodiments, the battery 220 may utilize disposable batteries, or can be recharged directly from a USB port 222 located on the interior or exterior of the body 210. The USB port 222 may further include USB connections such as: USB-A, USB-B, Micro-B, Micro-USB, Mini-USB, USB-C, etc. In addition, the interior of the body 210 may have a fumigation device or sprayer 230 that is also powered by the battery 220. The sprayer 230 contains one or more cartridge(s) 232 that can be filled with bed bug-killing or germ-treating chemicals or solutions, such as but not limited to: pyrethrins, pyrethroids, desiccants, cold-pressed neem oil, pyrroles, neonicotinoids, essential oils, diatomaceous earth, soapy solutions, vinegar based solutions, alcohol, witch hazel, bay leaves, cedar chips, citrus peels, chili powder, peppermint, spearmint, tea tree oil, lemongrass oil, eucalyptus oil and combinations thereof. In addition, the cartridge 232 may alternatively or additionally contain general disinfecting and cleaning products that may be scented. The sprayer 230 can further be activated via a button 234 that also allows a user to select the time interval in which the sprayer 230 will run. In this manner, the bedding set 100 can easily be sanitized while being transported within the bag 200 as a user travels. The bag may also contain a negative pressure device or vacuum 235 to help draw the various disinfecting and other treating compounds through the bedding set. The vacuum 235 is powered by a battery, as is the sprayer. A sensor 237 may also be present to determine whether sufficient treatment has occurred and whether the sensor detects any germs, pathogens or bugs.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “improved bedding set”, “betting set”, and “set”, are interchangeable and refer to the bedding set 100 of the present invention.

Notwithstanding the forgoing, the bedding set 100 of the present invention and its various components can be of any suitable size and configuration as is known in the art without affecting the overall concept of the invention, provided that they accomplish the above-stated objectives. One of ordinary skill in the art will appreciate that the size, configuration and material of the bedding set 100 as shown in the FIGS. are for illustrative purposes only, and that many other sizes and shapes of the bedding set 100 are well within the scope of the present disclosure. Although the dimensions of the bedding set 100 are important design parameters for user convenience, the bedding set 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A bedding set comprising:
a plurality of individual elements, wherein at least one of the plurality of individual elements is comprised of at least one of an anti-bacterial, an anti-viral or an anti-microbial component; and
a fastener used with at least two of the plurality of individual elements to secure the at least two individual elements to one another.

2. The bedding set as recited in claim 1, wherein the plurality of individual elements comprise at least a pillow case, a top sheet, a fitted sheet, a lining and a protective plastic layer.

3. The bedding set as recited in claim 2, wherein at least the lining and the protective plastic layer are provided with a fastener to secure the lining and the protective plastic layer together.

4. The bedding set as recited in claim 3, wherein the fastener is at least one of a snap, a clip, a hook and loop fastener, an adhesive fastener, a magnet fastener, a string, a zipper assembly or a combination thereof.

5. The bedding set as recited in claim 4, wherein the fastener is comprised of a first type of fastener that is used for an upper part of the bedding set and a second type of fastener is used for the a lower part of the bedding set.

6. The bedding set as recited in claim 5, wherein the first type of fastener is a fabric hook and loop fastener and the second type of fastener is a rigid fastener.

7. The bedding set as recited in claim 6, wherein second type of fastener is one of a snap, a magnet or a zipper.

8. The bedding set as recited in claim 1, wherein the at least one of an anti-bacterial, an anti-viral or an anti-microbial component is selected from a group consisting of a pyrethrin, a pyrethroid, a desiccant, a cold-pressed neem oil, a pyrrole, a neonicotinoids, an essential oil, a diatomaceous earth, a soapy solution, a vinegar-based solution, an alcohol, a witch hazel, a bay leave, a cedar oil, a citrus oil, a chili powder, a peppermint oil, a spearmint oil, a tea tree oil, a lemongrass oil, an eucalyptus oil and a combination thereof.

9. The bedding set as recited in claim 2, wherein the plurality of individual elements further comprise a duvet and a strap.

10. The bedding set as recited in claim 1, wherein the lining is manufactured from a memory foam material.

11. A combination linen set and carrying bag comprising:
a linen set comprising a pillowcase, a top sheet, a fitted sheet, a lining and a protective plastic layer; and
a carrying bag sized and configured to carry the linen set;
a sprayer; and
a UV light, wherein the sprayer and the UV light are attached to the carrying bag.

12. The combination linen set and carrying bag as recited in claim 11, wherein the sprayer and UV light are powered by a battery.

13. The combination linen set and carrying bag as recited in claim 12 further comprising a vacuum.

14. The combination linen set and carrying bag as recited in claim 12, wherein the sprayer comprises at least one cartridge for dispensing a solution to treat the linen set.

15. The combination linen set and carrying bag as recited in claim 13, wherein the cartridge includes at least one of a pyrethrin, a pyrethroid, a desiccant, a cold-pressed neem oil, a pyrrole, a neonicotinoid, an essential oil, a diatomaceous earth, a soapy solution, a vinegar based solution, an alcohol, a witch hazel, a bay leave, a cedar oil, a citrus oil, a chili powder, a peppermint oil, a spearmint oil, a tea tree oil, a lemongrass oil, an eucalyptus oil or a combination thereof.

16. The combination linen set and carrying bag as recited in claim 11, wherein the carrying bag comprises a sachet.

17. The combination linen set and carrying bag as recited in claim 11, wherein at least two of the pillowcase, the top sheet, the fitted sheet, the lining and the protective plastic layer comprise a fastener.

18. A bedding configuration comprising:
a mattress;
a linen set comprising a pillowcase, a top sheet, a fitted sheet, a lining, a duvet and a protective plastic layer; and
a travel bag sized and configured to hold the linen set, the travel bag comprising a UV light and a sprayer for dispensing a solution to treat germs, viruses, bugs and vermin, wherein at least two components of the linen set comprise a fastener to secure the at least two components together.

19. The bedding configuration as recited in claim 18 further comprising a strap for placement under the mattress and connection to the fasteners of the at least two components of the linen set.

20. The bedding configuration as recited in claim 18 further comprising a sensor positioned in the travel bag for detecting the germs, viruses, bugs and vermin.
